(19) United States
(12) Patent Application Publication
(10) Pub. No.: US 2023/0174599 A1
(43) Pub. Date: Jun. 8, 2023

(54) HIGH AFFINITY VIRAL CAPTURE HUMAN DECOY BASED PROTEINS FOR DETECTION AND PROTECTION AGAINST SARS-CoV-2 AND ZOONOTIC THREATS

(71) Applicant: The Regents of the University of California, Oakland, CA (US)

(72) Inventors: Michael D. TONEY, Davis, CA (US); Daniel L. COX, San Francisco, CA (US)

(21) Appl. No.: 17/921,303

(22) PCT Filed: Apr. 29, 2021
(86) PCT No.: PCT/US2021/029964

§ 371 Date: Oct. 25, 2022

Related U.S. Application Data
(60) Provisional application No. 63/018,285, filed on Apr. 30, 2020.

(51) Int. Cl. C07K 14/47; C07K 14/435; G01N 33/68; C07K 17/12; C12N 15/62

(52) U.S. Cl. CPC C07K 14/4711; C07K 14/43563; C07K 17/12; C12N 15/62; G01N 33/6896

(57) ABSTRACT

Amyloid fibrils comprising pathogen binding proteins and methods of their use are provided.

### CROSS REFERENCE TO RELATED PATENT APPLICATIONS

The present application is a U.S. National Phase Application Under 371 of International Application PCT/US2021/029964 filed Apr. 29, 2021, which claims benefit of priority to U.S. Provisional Pat. Application No. 63/018,285, filed Apr. 30, 2020, each of which is incorporated by reference for all purposes.

### SEQUENCE LISTING

The instant application contains a Sequence Listing which has been submitted electronically in ASCII format and is hereby incorporated by reference in its entirety. Said ASCII copy, created on Jun. 15, 2021, is named 081906-1245167-238310PC_SL.txt and is 22,858 bytes in size.

### BACKGROUND OF THE INVENTION

The decoy vs. antibody approach[1,2,3] is a fundamental shift in viral diagnostic strategy. Zoonotic endemic pandemic threats require a universal solution which protects against the steady pace of evolutionary mutational variants from an original wild type crossover virus. The current focus is on SARS-CoV-2, first seen in Wuhan. This decoy approach vs. antibody approach represents a standard which can be employed against future zoonotic events in antigen assay diagnostics.

### BRIEF SUMMARY OF THE INVENTION

Our invention creates high affinity human receptor decoys for diagnostics. It replicates the point of infection (ACE2 receptor) and detects the virus by directly capturing virions from solution with efficacy that will not degrade as the virus mutates from wildtype. Multivalency leads to a binding affinity which can be up to 1,000,000 times stronger than ACE2 alone, and which can lead to dramatic improvements in diagnostic sensitivity, lowering of detection thresholds, and expansion of time windows for detection. These proteins are environmentally stable and inexpensively produced in standard microbial expression systems, offering further advantages over antibodies in antigen based diagnostic assays.

In some embodiments, an amyloid fibril comprising a plurality of modified β solenoid protein (mBSP) monomers is provided, wherein the monomers are linked to a pathogen-binding protein. In some embodiments, the mBSP monomers are derived from an antifreeze protein[4].

In some embodiments, the antifreeze protein is a spruce budworm antifreeze protein. In some embodiments, the mBSP has the sequence shown in SEQ ID NO: 1 or a sequence at least 90%, 95%, or 98% identical to SEQ ID NO: 1.

In some embodiments, the antifreeze protein is a rye grass antifreeze protein. In some embodiments, the mBSP has the sequence shown in SEQ ID NO: 2 or a sequence at least 90%, 95%, or 98% identical to SEQ ID NO: 2.

In some embodiments, the antifreeze protein is a rhagium inquisitor antifreeze protein. In some embodiments, the mBSP has the sequence shown in SEQ ID NO: 3 or a sequence at least 90%, 95%, or 98% identical to SEQ ID NO: 3.

In some embodiments, mBSP is modified to remove an end cap that prevents amyloid aggregation.

In some embodiments, the amyloid fibril is modified to include at least one amino acid residue that promotes attachment of the fibril to a solid support, a nanoparticle, a biological molecule, or a second amyloid fibril.

In some embodiments, the amyloid fibril is attached to a solid support, a nanoparticle, a biological molecule, or a second amyloid fibril.

In some embodiments, the pathogen is a virus. In some embodiments, the virus is SARS-COV-2 or HBV or HCV.

In some embodiments, the amyloid fibril is linked to a solid support. In some embodiments, the solid support is absorbent. In some embodiments, the solid support is paper.

In some embodiments, the pathogen-binding protein comprises the N-terminal ACE2 helix-turn-helix (HTH) domain (e.g., SEQ ID NO: 4 or a sequence at least 90%, 95%, or 98% identical to SEQ ID NO: 4).

In some embodiments, the pathogen binding protein is linked to a detectable label In some embodiments, the detectable label changes signal depending on whether the pathogen-binding protein is binding the pathogen.

Also provided is a method of detecting the presence or absence of a pathogen in a biological sample. In some embodiments, the method comprises contacting a biological sample with the amyloid fibril as described above or elsewhere herein under conditions that allow the pathogen to bind to the pathogen binding protein if the pathogen is present; and detecting the presence or absence of binding of the pathogen to the amyloid fibril. In some embodiments, the amyloid fibril is linked to a detectable label that changes signal depending on whether the pathogen-binding protein is binding the pathogen, and detection comprises detecting signal from the detectable label. In some embodiments, the detecting comprises washing unbound components of the sample from the amyloid fibril and contacting the amyloid fibril, and bound pathogen if present, with a secondary binding agent that specifically binds to the pathogen, if bound to the amyloid fibril.

In some embodiments, the secondary binding agent is a β solenoid protein linked to a pathogen binding protein. In some embodiments, the secondary binding agent is linked to a detectable label. In some embodiments, the β solenoid protein comprises a sequence that prevents polymerization.

Also provided is clothing or protective equipment coated with the amyloid fibril as described above or elsewhere herein.

### DEFINITIONS

The term “β-solenoid protein” (BSP) refers to proteins having backbones that turn helically in either a left- or right-handed sense around the long axis of the protein from the N-terminus to the C-terminus to form contiguous β-sheets, and have regular geometric structures (triangles, rectangles, etc.) with 1.5-2 nm sides. The wild type (WT) BSPs do not undergo end-to-end polymerization to give cross β-fibrils due to natural capping features and/or structural irregularities on one or both ends. Examples of non-amyloidogenic WT-BSPs that can form amyloid fibrils upon modification include, one-sided antifreeze proteins (Tenebrio molitor AFP- Protein Database (PDB) Accession No. 1EZG), two-sided antifreeze (Snow Flea AFP- PDB 2PNE and 3BOI), rye grass AFP (PDB- 3ULT), three-sided “type II” left handed β-helical solenoid antifreeze proteins, for example from the spruce budworm (PDB 1M8N), three-sided bacterial enzymes (PDB 1LXA, 1FWY, 1G95, 1HV9, 1J2Z, 1T3D, 1THJ, 1KGQ, 1MR7, 1SSM, 2WLC, 3R3R, 1KRV, 3EH0, 3Q1X, 3BXY, 3HJJ, 3OGZ, 4M98, 4IHH (acyltransferases, γ-class carbonic anhydrases and homologs), three-sided motor proteins subunits (e.g., PDB 3TV0), a three-sided “type I” left handed β-helical enzyme ydcK from Salmonellae cholera (2PIG), four-sided proteins (PDB 2BM6, 2W7Z, 2J8I), four-sided pentapeptide repeat proteins (2G0Y and 3DU1), and 1XAT. The full sequence of each of these proteins is available from the Protein Database.

The term “modified β solenoid protein (mBSP)” (also referred to as mBSP monomer) refers to genetically engineered β solenoid proteins that allow for amyloid self-assembly. A mBSP monomer can be engineered to be any desired length and can be tailored to the particular application. In a typical embodiment, the monomer will comprise at least two beta sheet rungs (about 30-36 residues) and more often at least three rungs (about 45-54 residues). The typical size of a beta strand face is about 3-6 residues, including bends the edge size will usually not exceed 5-8 residues, which is a range of about 2-3.2 nm. A number of modifications can be used to allow for self-assembly. For example, many BSPs include end caps that can be removed to allow for amyloid self-assembly. For example, in the spruce budworm antifreeze protein, the endcap inhibiting aggregation consists of a reverse in the helicity from left to right handed initiated at a bend at residue 102 and extending to the C-terminus at residue 121. This reversed helicity prevents precise epitaxy at the C-terminal to N-terminal interface of the wild type protein. The cap sequences in this case are:

RGVATPAAACKISGCSLSAM (SEQ ID NO: 10).

For the wild type ryegrass antifreeze protein, the removed cap sequence is the C-terminal layer

AAKLAAALEHHHHHH (SEQ ID NO: 11)(residues 119-133).

For the wild type rhagium inquisitor antifreeze protein, the first N-terminal layer has right handed helicity

(residues 1-19, GYSCRAVGVDGRAVTDIQG (SEQ ID NO: 12)),

while the presented, removed, top layer has left handed helicity, inhibiting epitaxy

(residues 107-126, QPTQTQTITGPGFQTAKSFA (SEQ ID NO: 13)).

In addition, many BSPs include disulfides, bulges, and prolines that require removal to allow for amyloid self-assembly. The three dimensional structure of any given BSP can be used to design an mBSP of that desired shape. Means for modeling engineered proteins and characterizing their final properties are well known to those of skill. Exemplary techniques for these procedures are described in, e.g., U.S. Pat. No. 10,287,332[5]. Examples of mBSPs include SBAFP-m9 (SBAFP with endcap and disulfides removed), and RGAFP-ml (RGAFP with bulges and proline removed), both of which are described in more detail below. A detailed description for the RiAFP-m9 can be found in Ref. [11].

The mBSPs can be functionalized in designed ways to specifically carry designated functional units, for example pathogen binding proteins, which are fused to the amino- or carboxyl- or both termini of mBSPs. In some embodiments, the monomers can further include one or more amino acid residues at the end. In other embodiments, the functional units can be inserted at the corners of the mBSP structures, within the sequence of the mBSP. The residues can be selected to allow attachment of the mBSP or fibril to a solid support, a nanoparticle, a biological molecule (e.g., an enzyme), a bacterial or eukaryotic cell, or additional amyloid fibrils. For example, the mBSP monomers can be modified to include residues that enhance hydrophobic interactions and/or salt bridging. Peptide bond chemistry, threonine bonding, disulfide bridges, or metal mediated chelation of histidine side chains can also be used. By adjusting the side chain structures on different faces of mBSPs, programmable assembly of two and three dimensional structures can be achieved. Modifications of external side chains of the mBSPs can be used to enable binding to solid supports, or for specific lateral self-assembly.

The term “amyloid fibril” refers to fibrous proteins that polymerize end-to-end in one-dimensional protein arrays. Amyloid fibrils can form naturally or they can be produced out of intrinsically non-amyloidogenic proteins. As shown here, using a rational design concept, intrinsically non-amyloidogenic proteins (e.g., BSPs) with natural cross-β structure can be transformed into proteins that readily self-assemble into amyloid fibrils under benign conditions.

The term “mBSP scaffold” refers to a system of one or more amyloid fibrils comprising mBSP monomers, that can be a platform for biomaterial-based self-assembly.

The term “antifreeze protein or AFP” refers to a protein found in the body fluids of some poikilothermic organisms, such as, Choristoneura sp. C. fumiferana or C. occidentalis, the Tenebrio molitor mealworm and plants which have the commonly known property that they reduce non-colligatively the freezing point of water. As used herein, “antifreeze proteins” are chemically synthesized or recombinantly produced polypeptides having a protein sequence with substantial similarity to a naturally occurring antifreeze protein and retaining the properties of an antifreeze polypeptide. In some embodiments, the modified antifreeze proteins will have altered or improved antifreeze activity and can be used for that purpose, as well.

Many antifreeze proteins are BSPs. For example, those derived from Tenebrio, Snow Flea rye grass, and the spruce budworm. Other examples of antifreeze proteins useful in the present invention include those described in the following PDB Accessions: 3VN3_B, 3VN3_A, 4DT5_B, and 4DT5_A.

The terms “identical” or percent “identity,” in the context of two or more nucleic acids or polypeptide sequences, (e.g., two mBSPs and polynucleotides that encode them) refer to two or more sequences or subsequences that are the same or have a specified percentage of amino acid residues or nucleotides that are the same, when compared and aligned for maximum correspondence, as measured using one of the following sequence comparison algorithms or by visual inspection.

The phrase “substantially identical,” in the context of two nucleic acids or polypeptides of the invention, refers to two or more sequences or subsequences that have at least 60%, 65%, 70%, 75%, 80%, or 90-95% nucleotide or amino acid residue identity (e.g., to any of the sequences here, including but not limited to SEQ ID NO: 1, 2, and 3), when compared and aligned for maximum correspondence, as measured using one of the following sequence comparison algorithms or by visual inspection. Preferably, the substantial identity exists over a region of the sequences that is at least about 50 residues in length, more preferably over a region of at least about 100 residues, and most preferably the sequences are substantially identical over at least about 150 residues. In a most preferred embodiment, the sequences are substantially identical over the entire length of the coding regions.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat’l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F.M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschuel et al. (1977) Nucleic Acids Res. 25: 3389-3402, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always > 0) and N (penalty score for mismatching residues; always < 0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=-4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

“Biological sample” includes tissues and bodily fluids, e.g., blood, blood fractions, lymph, saliva, urine, feces, etc.

The terms “specific for,” “specifically binds,” and like terms refer to a molecule (e.g., antibody or antibody fragment) that binds to a target with at least 2-fold greater affinity than non-target compounds, e.g., at least any of 4-fold, 5-fold, 6-fold, 7-fold, 8-fold, 9-fold, 10-fold, 20-fold, 25-fold, 50-fold, or 100-fold greater affinity. Specificity can be determined using standard methods, e.g., solid-phase ELISA immunoassays (see, e.g., Harlow & Lane, Using Antibodies, A Laboratory Manual (1998) for a description of immunoassay formats and conditions that can be used to determine specific immunoreactivity).

A “label” or a “detectable label” is a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known for conjugating a protein to the label may be employed, e.g., using methods described in Hermanson, Bioconjugate Techniques 1996, Academic Press, Inc., San Diego.

### BRIEF DESCRIPTION OF THE FIGURES

FIG. 1 Example sequence comparisons of wild type (WT) anti-freeze proteins to modified proteins that allow for self-assembly. In each case, C-terminal end cap sequences are removed to allow C-to-N-terminal amyloid assembly. Additionally, modifications are made throughout to allow for solubility, stability, and microbial expression as detailed in [0016-0018]. FIG. 1 discloses SEQ ID NOS 7, 1, 8, 2, 9, and 3, respectively, in order of appearance.

FIG. 2 Example structural comparison of wild type (WT) RiAFP to RiAFP-m9. Removed end cap region of WT is highlighted in lighter shade.

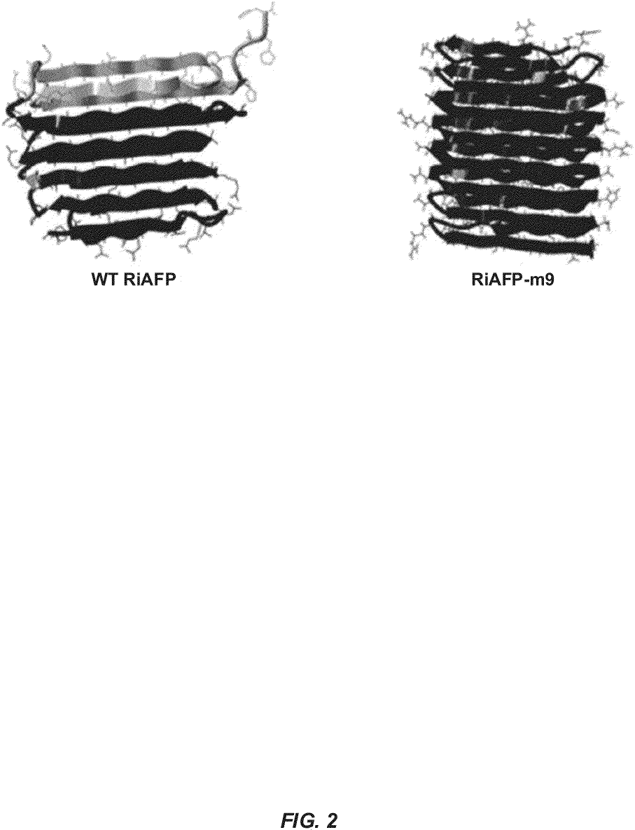

FIG. 3 Sample mBSP derived from modified Rhagium inquisitor[2] antifreeze protein (RiAFP). (Top) An engineered VEGF binding domain is shown on the right. The flat faces and the polypeptide termini can readily be modified to provide for attachment to surfaces or covalent attachment of reporter molecules such as fluorophores. (Bottom) The mBSPs are engineered to polymerize under mild conditions to provide a high degree of polyvalency and thus avidity.

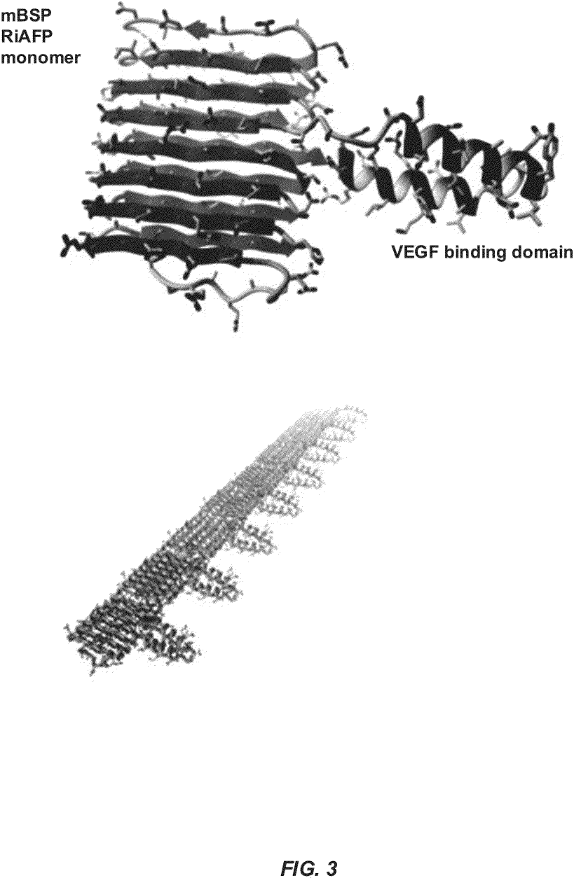

FIG. 4 Model of the structure of the proposed mBSP-ACE2 HTH fusion for SARS-CoV-2 virion capture. (Top) The N-terminal HTH motif of the ACE2 structure fused to RiAFP mBSP with the SARS-CoV-2 spike protein shown bound. This structure is stable in simulations over ~10 ns at 100 C. (Bottom) Overlay of the RiAFP-ACE2 HTH fusion with the experimental structure for the ACE2-Spike protein complex, illustrating why the N-terminal HTH motif was chosen for SARS-CoV-2 capture.

FIG. 5. Specific designs of mBSP-HTH constructs used to obtain affinity data for this patent. A) mBSP-HTH.1 Design - HTH is fused at a bend of modified Rhagium inquisitor antifreeze protein [SEQ ID NO: 5]. B) mBSP-HTH.2 Design - HTH with added spacing peptide sequences are fused at a bend of modified Rhagium inquisitor antifreeze protein [SEQ ID NO: 6].

FIG. 6. Table of measured dissociation constant values for primary bound protein RBD at tip and ligand proteins with HTH. Comparison with two other ACE2 N-terminal mimics from literature shown.

FIG. 7 Schematic mBSP assay for SARS-CoV-2 detection.

FIG. 8. Schematic for fluorescent assay demonstrating polyvalent binding. A) Designed viral mimic nanoparticle. Spike RBD proteins are attached to the surface of fluorescent nanoparticles to assay binding to polymeric mBSP-HTH constructs. B) Polymeric mBSP-HTH constructs are fixed to nitrocellulose followed by dried milk to coat empty space on nitrocellulose.

FIG. 9. Fluorescent Assay to demonstrate polyvalent binding of SARS-CoV-2 Spike RBDs by mBSP-HTH constructs. A) Control: BSA protein on nitrocellulose shows no fluorescence, indicating no binding of fluorescent nanoparticles, B) Image 24 hours after deposition shows fluorescence on two separate patches where mBSP-HTH.2 proteins have been deposited. C) Image 24 hours later shows little decrease in fluorescent intensity consistent with polyvalent binding.

FIG. 10. Summary of dimeric RBD capture constructs based upon ACE2 or decoys, including results from two other groups.

FIG. 11 Proof of principle VEGF binding experiment with engineered mBSP RiAFP fibrils.

### DETAILED DESCRIPTION OF THE INVENTION

The inventors have discovered that amyloid fibrils comprised of monomers linked to pathogen binding proteins can be used to effectively detect pathogens. Amyloid fibrils are of particular use because they are stable under many conditions and can present a high density of binding molecules allowing for high affinity binding to pathogens. Amyloid fibrils can be formed from a number of β solenoid proteins that can be modified to remove a naturally-occurring motif that blocks polymerization. By fusing a pathogen binding protein to one, some or all of the β solenoid protein monomers, and allowing the monomers to polymerize, one can form a polymer with numerous pathogen binding sites. The polymer can be optionally linked to a solid support and can be used to detect pathogens in samples.

Naturally occurring β-solenoid proteins (BSPs) can be modified to form amyloid fibrils. These proteins have backbones that turn helically in either a left- or right-handed sense from the N-terminus to form β-sheets, and have regular geometric structures (triangles, rectangles, etc.) with 1.5-2 nm sides. The naturally occurring proteins are inhibited from amyloid aggregation (end-to-end polymerization to give cross β-fibrils) by natural capping features and/or structural distortions on one or both ends. Modification for making linear polymers (amyloids) from these proteins, molecular simulations used to assess structural stability and geometric properties for comparison to measurements, and the protocol for expressing and folding of the engineered proteins are described in, e.g., U.S. Pat. No. 10,287,332, which is incorporated by reference[5]. The correct monomeric structures can be obtained after purification and folding, amyloid fibrils can be produced by incubation at elevated temperatures, and the kinetics of fibril formation are consistent with, though slightly faster than, other amyloid polymerization reactions.

The modified BSPs (mBSPs) offer excellent platforms for functionalization with pathogen binding proteins without interfering with the native β-sheet structure. This allows for presentation of a number of the same or different pathogen binding proteins while maintaining the beta-sheet.

In one embodiment, the BSP is modified to enable one-dimensional growth through cross-beta strand (amyloid) pairing mBSPs. The exteriors and interiors of the proteins can also be modified to enable more efficient production. In some embodiments, the protein units are allowed to self-assemble in one dimension after expressing proteins (for example but not limited to, in E. coli), followed by, for example, subsequent cell lysis, purification, denaturation, refolding, and polymerization of the proteins to create the one dimensional scaffolds.

In some embodiments, at least two different mBSP monomers are designed to self-assemble in a predetermined order. This can be achieved by modifying the ends of the monomers such that, for example, the N-terminus of a first monomer interfaces with the C-terminus of a second monomer, but not with the C-terminus of another copy of the first monomer. The resulting fibril comprises the two different monomers in predetermined order (e.g., A-B-A-B-A- B, or A-B-C-A-B-C). For example, A, B, and C can represent monomers fused to different pathogen binding proteins, where different pathogen binding proteins have different sequences but bind to the same pathogen (optionally to different locations of the same pathogen) or bind to different pathogens.

The correct molecular mass of the amyloid monomer can be verified through standard techniques, such as mass spectroscopy. The correct beta content can be determined through techniques such as circular dichroism. Amyloid aggregation can be confirmed by observing the growth of thioflavin T (ThT) fluorescence at 480 nm.

The length of the fibrils can be controlled, for example through a variety of approaches including varying of the temperature (e.g., between 5° C. to 45° C.), by following the incubation with sonication, by the addition of inhibitors of polymerization, or by modifying the buffer solution. For example, fibrils of several microns can be routinely produced. Alternatively, shorter fibrils (e.g., 100-200 nm) can be produced upon sonication.

Exemplary naturally-occurring BSPs include but are not limited to:

1. Chain A, Crystal Structure Of A Lumenal Pentapeptide Repeat Protein From Cyanothece Sp 51142 At 2.3 Angstrom Resolution. Tetragonal Crystal Form

PDB: 2G0Y A

1 mhhhhhhssg lvprgsgmke taakferqhm dspdlgtddd dkamamvtgs sasyedvkli
61 gedfsgkslt yaqftnadlt dsnfseadlr gavfngsali gadlhgadlt nglayltsfk
121 gadltnavlt eaimmrtkfd dakitgadfs lavldvyevd klcdradgvn pktgvstres
181 lrcq (SEQ ID NO: 14)

2. Chain X, The 2.0 Angstrom Resolution Crystal Structure Of Hetl, A Pentapeptide Repeat Protein Involved In Heterocyst Differentiation Regulation From The Cyanobacterium Nostoc Sp. Strain Pcc 7120

PDB: 3DU1_X

1 mgsshhhhhh ssglvprgsh mnvgeilrhy aagkrnfqhi nlqeieltna sltgadlsya
61 dlrqtrlgks nfshtclrea dlseailwgi dlseadlyra ilreadltga klvktrleea
121 nlikaslcga nlnsanlsrc llfqadlrps snqrtdlgyv lltgadlsya dlraaslhha
181 nldgaklcra nfgrtiqwgn laadlsgasl qgadlsyanl esailrkanl qgadltgail
241 kdaelkgaim pdgsihd (SEQ ID NO: 15)

3. Chain A, Crystal Structure Of Recombinant Human Alpha Lactalbumin

PDB: 3B0I_A

1 mkqftkcels qllkdidgyg gialpelict mfhtsgydtq aivenneste yglfqisnkl
61 wckssqvpqs rnicdiscdk flddditddi mcakkildik gidywlahka lctekleqwl
121 cekl (SEQ ID NO: 16)

4. Chain B, Crystal Structure Of An Ice-Binding Protein From The Perennial Ryegrass, Lolium Perenne

PDB: 3ULT ₋B

1 mdeqpntisg snntvrsgsk nvlagndntv isgdnnsvsg snntvvsgnd ntvtgsnhvv
61 sgtnhivtdn nnnvsgndnn vsgsfhtvsg ghntvsgsnn tvsgsnhvvs gsnkvvtdaa
121 klaaalehhh hhh (SEQ ID NO: 8)

5. Chain A, Crystal Structure Of An Ice-Binding Protein From The Perennial Ryegrass, Lolium Perenne

PDB: 3ULT_A

1 mdeqpntisg snntvrsgsk nvlagndntv isgdnnsvsg snntvvsgnd ntvtgsnhvv
61 sgtnhivtdn nnnvsgndnn vsgsfhtvsg ghntvsgsnn tvsgsnhvvs gsnkvvtdaa
121 klaaalehhh hhh (SEQ ID NO: 8)

6. Chain B, Crystal Structure Of Ydck From Salmonella Cholerae At 2.38 A Resolution. Northeast Structural Genomics Target Scr6

PDB: 2PIG_B

1 xtkyrlsegp raftyqvdge kksvllrqvi avtdfndvka gtsggwvdad nvlsqqgdcw
61 iydenaxafa gteitgnari tqpctlynnv rigdnvwidr adisdgaris dnvtiqsssv
121 reecaiygda rvlnqseila iqglthehaq ilqiydratv nhsrivhqvq lygnatitha
181 fiehraevfd faliegdkdn nvwicdcakv ygharviagt eedaiptlry ssqvaehali
241 egncvlkhhv lvgghaevrg gpillddrvl ieghaciqge ilierqveis graaviafdd
301 ntihlrgpkv ingedritrt plvgsllehh hhhh (SEQ ID NO: 17)

7. Chain A, Crystal Structure Of Ydck From Salmonella Cholerae At 2.38 A Resolution. Northeast Structural Genomics Target Scr6

PDB: 2PIG_A

1 xtkyrlsegp raftyqvdge kksvllrqvi avtdfndvka gtsggwvdad nvlsqqgdcw
61 iydenaxafa gteitgnari tqpctlynnv rigdnvwidr adisdgaris dnvtiqsssv
121 reecaiygda rvlnqseila iqglthehaq ilqiydratv nhsrivhqvq lygnatitha
181 fiehraevfd faliegdkdn nvwicdcakv ygharviagt eedaiptlry ssqvaehali
241 egncvlkhhv lvgghaevrg gpillddrvl ieghaciqge ilierqveis graaviafdd
301 ntihlrgpkv ingedritrt plvgsllehh hhhh (SEQ ID NO: 17)

8. Chain A, Choristoneura Fumiferana (Spruce Budworm) Antifreeze Protein Isoform 501

PDB: 1M8N_A

1 dgtcvntnsq itansqcvks tatncyidns qlvdtsictr sqysdanvkk svttdcnidk
61 sqvylttctg sqyngiyirs stttgtsisg pgcsistcti trgvatpaaa ckisgcslsa
121 m (SEQ ID NO: 7)

9. Chain B, Choristoneura Fumiferana (Spruce Budworm) Antifreeze Protein Isoform 501

PDB: 1M8N_B

1 dgtcvntnsq itansqcvks tatncyidns qlvdtsictr sqysdanvkk svttdcnidk
61 sqvylttctg sqyngiyirs stttgtsisg pgcsistcti trgvatpaaa ckisgcslsa
121 m (SEQ ID NO: 7)

10. Chain C, Choristoneura Fumiferana (Spruce Budworm) Antifreeze Protein Isoform 501

PDB: 1M8N_C

1 dgtcvntnsq itansqcvks tatncyidns qlvdtsictr sqysdanvkk svttdcnidk
61 sqvylttctg sqyngiyirs stttgtsisg pgcsistcti trgvatpaaa ckisgcslsa
121 m (SEQ ID NO: 7)

11. Chain D, Choristoneura Fumiferana (Spruce Budworm) Antifreeze Protein Isoform 501

PDB: 1M8N_D

1 dgtcvntnsq itansqcvks tatncyidns qlvdtsictr sqysdanvkk svttdcnidk
61 sqvylttctg sqyngiyirs stttgtsisg pgcsistcti trgvatpaaa ckisgcslsa
121 m (SEQ ID NO: 7)

Exemplary mBSPs include but are not limited to:

SEQ ID NO:1 synthetic protein SBAFP-m9

Ala Ser Arg Ile Thr Asn Ser Gln Ile Val Lys Ser Glu Ala Thr
Asn
1 5 10 15
Ser Asp Ile Asn Asn Ser Gln Leu Val Asp Ser Ile Ser Thr Arg
Ser
20 25 30
Gln Tyr Ser Asp Ala Asn Val Lys Lys Ser Val Thr Thr Asp Ser
Asn
35 40 45
Ile Asp Lys Ser Gln Val Tyr Leu Thr Thr Ser Thr Gly Ser Gln
Tyr
50 55 60
Asn Gly Ile Tyr Ile Arg Ser Ser Asp Thr Thr Gly Ser Glu Ile
Ser
65 70 75 80
Gly Ser Ser Ile Ser Thr Ser Arg Ile Thr Ile
85 90

SEQ ID NO:2 synthetic protein RGAEP-m1

Ala Asn Asp Ile Asp Gly Thr Asn Asn Glu Val Asp Gly Ser Glu
Asn
1 5 10 15
Val Leu Ala Gly Asn Asp Asn Thr Val Ser Gly Asp Asn Asn Ser
Val
20 25 30
Ser Gly Ser Asn Asn Thr Val Ser Gly Asn Asp Asn Thr Val Thr
Gly
35 40 45
Ser Asn Met Val Val Ser Gly Thr Asn Met Ile Val Thr Asp Asn
Asn
50 55 60
Asn Asn Val Ser Gly Asn Asp Asn Asn Val Ser Gly Ser Phe Met
Thr
65 70 75 80
Val Ser Gly Gly Met Asn Thr Val Ser Gly Ser Asn Asn Thr Val
Ser
85 90 95
Gly Lys Arg Met Arg Val Gln Gly Thr Asn Asn Arg Val Thr Asp
100 105 110

SEQ ID NO: 3 synthetic protein RiAFP-m9

Ser Arg Ala Glu Ala Arg Gly Glu Ala Met Ala Glu Gly His Ser
Arg
5 10 15
Gly Cys Ala Thr Ser His Ala Asn Ala Thr Gly His Ala Asp Ala
Arg
10 15 20
Ser Met Ser Glu Gly Asn Ala Glu Ala Tyr Thr Glu Ala Lys Gly
Thr
25 30 35
Ala Met Ala Thr Ser Glu Ala Ser Gly Glu Ala Arg Ala Gln Thr
Asn
40 45 50
Ala Asp Gly Arg Ala His Ser Ser Ser Arg Thr His Gly Arg Ala
Asp
55 60 65 70
Ser Thr Ala Ser Ala Lys Gly Glu Ala Met Ala Glu Gly Thr Ser
Asp
75 80 85
Gly Asp Ala Lys Ser Tyr Ala Ser Ala Asp Gly Asn Ala Cys Ala
Lys
90 95 100
Ser Met Ser Thr Gly His Ala Asp Ala Thr Thr Asn Ala His Gly
Thr
105 110 115
Ala Met Ala Asp Ser Asn Ala Ile Gly Glu Ala Arg Ala Glu Thr
Arg
120 125 130
Ala Glu Gly Arg Ala Glu Ser Ser Ser Asp Thr Asp Gly Cys
135 140 145

In some embodiments, binding of scaffolds to solid support (e.g., surface) can be achieved. For example, in some embodiments, binding can be achieved by: (a) sulfur chemistry of unoxidized cysteine to bind to thiols decorating a prepared surface; (b) peptide bond chemistry to link exposed lysine side chains to carboxyl groups decorating a prepared solid surface; and (c) the application of common types of bioconjugate chemistry, for example the biotin-avidin or biotin-streptavidin interacting pair.

In certain circumstances, the solid support can be mica, silicon, glass, or a transparent conducting oxide, for example, FTO or ITO. In some embodiments, the surface can be poly-L-lysine coated mica (0001) surfaces. In other embodiments, the solid support is absorbent. For example, the solid support can be paper. This will allow for collection of a liquid biological sample, for example, saliva, blood, urine, feces, waste water or other biological fluids.

As noted above, the BSP or mBSP is fused to a pathogen binding protein, which provides an affinity agent that is displayed in multiple copies in the amyloid fibril polymer. The fusion can be direct between the monomer and the pathogen binding protein or a linker can be used to link the two fusion partners. In some embodiments, the linker can be comprised of a majority or entirely of glycine, serine or alanine or combinations thereof. For example, an exemplary linker is GGG.

The pathogen binding protein can be any protein that can be formed in a translational fusion with a BSP or mBSP monomer. The pathogen binding protein will depend on the pathogen to be targeted. In some embodiments, the pathogen binding protein is a protein from a human or animal or plant cell to which the pathogen binds during infection, and thus has affinity for the pathogen. In the example of SARS-CoV-2, the virus enters cells by binding Angiotensin Converting Enzyme 2 (ACE2). Accordingly, a useful SARS-CoV-2 binding protein is the virus-binding portion of ACE2. In some embodiments, the SARS-CoV-2 binding protein comprises a 54 residue helix-turn-helix (HTH) motif from ACE2, for example

IEEQAKTFLDKFNHEAEDLFTQSSLASTNTNTNITEENVQNMNNAGDKTS
AFLKEQSTLAQMT (SEQ ID NO:4)

or an amino acid sequence substantially identical to SEQ ID NO:4. Alternatively, the pathogen binding protein can be a peptide (e.g., identified by phage panning or other techniques), a tetrameric, single domain or single chain antibody, or other protein that selectively binds to the pathogen. Aside from SARS-CoV-2, the pathogen can be any virus, bacterium, protozoan, or fungus.

In some embodiments the combined binding protein-BSP fusion construct may be comprised from the 233 residue RiAFP and helix-turn-helix sequence (RiAFP-HTH.1)

ASRAEARGEAMAEGHSRGSATSHANATGHADARSMSEGNAEAYTEAKGDA
MATSEASGEARAQTNADGSAHSSSRTHGRADSTASAKTNYNRECGEEQAK
TFLDKFNHEAEDLFYQSSLASWNYNTNITEENVQNRNNAGDKRSAFLKEQ
STLAQMYGCGSGSAMAEGTSDGDAKSYASADGNASAKSMSTGHADATTNA
HGTAMADSNAIGEARAETRAEGRAESSSDTDGS (SEQ ID NO:5)

or an amino acid sequence substantially identical to SEQ. ID NO:5. In some embodiments the combined binding protein-BSP fusion construct may be comprised from the 266 residue RiAFP and helix-turn-helix sequence (RiAFP-HTH.2)

ASRAEARGEAMAEGHSRGSATSHANATGHADARSMSEGNAEAYTEAKGDA
MATSEASGEARAQTNADGSAHSSSRTHGRADSTASAKGGKALNDKEAKNK
AILNLEEIEEQAKTFLDKFNHEAEDLFYQSSLASWNYNTNITEENVQNMN
NAGDKWSAFLKEQSTLAQMYQNEIKRKSEKQEDLKKEMLELEKLGGAMAE
GTSDGDAKSYASADGNASAKSMSTGHADATTNAHGTAMADSNAIGEARAE
TRAEGRAESSSDTDGS (SEQ ID NO:6)

or an amino acid sequence substantially identical to SEQ ID NO:6. In this embodiment the helix-turn-helix on each side is flanked by spacing linkers so it sticks out further from the mBSP scaffold.

In some embodiments, the pathogen is a hepatitis virus, e.g., hepatitis A, hepatitis B (HBV) or hepatitis C (HCV). Exemplary HBV binding proteins include but are not limited to the heptapeptide

ETGAKPH (SEQ ID NO: 18)

(interacting with the hydrophilic loop (residues 101-159 located on the surface of the virus) with the dissociation constant of 2.9 nM [Ho, K.L., et al., Selection of high affinity ligands to hepatitis B core antigen from a phage-displayed cyclic peptide library. J Med Virol, 2003. 69(1): p. 27-32]). Exemplary HCV binding proteins include but are not limited to

TSQNIRS (SEQ ID NO: 19)

,_which binds to the Hepatitis C Virus Protein E2 [Hong, H.W., S.W. Lee, and H. Myung, Selection of peptides binding to HCV e2 and inhibiting viral infectivity. J Microbiol Biotechnol., 2010. 20(12): p. 1769-71] and

WPWHNHR (SEQ ID NO: 20)

Lu, X., et al., Identification of peptides that bind hepatitis C virus envelope protein E2 and inhibit viral cellular entry from a phage-display peptide library, in Int J Mol Med. 2014, 2011 Elsevier Inc: Greece] and

MARHRNWPLVMV (SEQ ID NO: 21)

[Chen, F., et al., Functional selection of hepatitis C virus envelope E2-binding Peptide ligands by using ribosome display. Antimicrob Agents Chemother., 2010. 54(8): p. 3355-64. doi: 10.1128/AAC.01357-09. Epub 2010 May 17].

In some embodiments, the amyloid fibril is composed of two or more mBSP monomer/pathogen binding protein fusions, wherein the two or more fusions differ by the pathogen binding protein. Thus, in some embodiments, the amyloid fibril comprises two or more different pathogen binding proteins that bind to the same pathogen, optionally at different targets on the pathogen. In some embodiments, the different pathogen binding proteins bind to different pathogens.

As noted above, the amyloid fibrils comprising pathogen binding proteins can be linked to a solid support, for example an absorbent solid support (e.g., paper or other cellulose or other polymer material), which can capture biological fluids. In some embodiments, the amyloid fibrils comprising pathogen binding proteins are linked to an absorbent solid support and used in detection of pathogen using lateral flow.

In other embodiments, the amyloid fibrils comprising pathogen binding proteins can be embedded in or coated on personal protective equipment (PPE). Exemplary PPE include but are not limited to clothing (e.g., lab gowns or jackets or hazmat suits), gloves, face masks, face screens or other physical barriers. In some embodiments, the amyloid fibrils comprising pathogen binding proteins can be formulated into a liquid, gel, or cream that can be applied to surfaces (e.g., countertops) or to the skin to bind and inactivate pathogen, if present, preventing the pathogens from being infectious.

In addition to binding pathogens to inactivate them, the amyloid fibrils comprising pathogen binding proteins can be used to capture and detect a pathogen. In some embodiments, the amyloid fibrils comprising pathogen binding proteins are linked to a solid support as described herein and contacted with a sample (e.g., a biological sample); pathogen specifically bound to the pathogen binding protein can be detected. In some embodiments, the bound pathogen is detected by a sandwich assay format, with a second binding agent binding the immobilized pathogen and subsequent detection of the bound second binding agent. The second binding agent can be labeled or can be contacted by a tertiary binder that comprises a detectable label. In some embodiments, the second binding agent is a mBSP comprising a cap sequence that prevents polymerization fused to a pathogen binding protein. In some embodiments, the second binding agent can be a peptide (e.g., 10-50 amino acids) or an antibody that specifically binds the pathogen. Alternatively, the amyloid fibrils comprising pathogen binding proteins can be linked to a detectable label that changes the signal depending on whether the pathogen is bound to the pathogen binding protein, thereby avoiding the need for a second binding agent.

### EXAMPLES

### Example 1

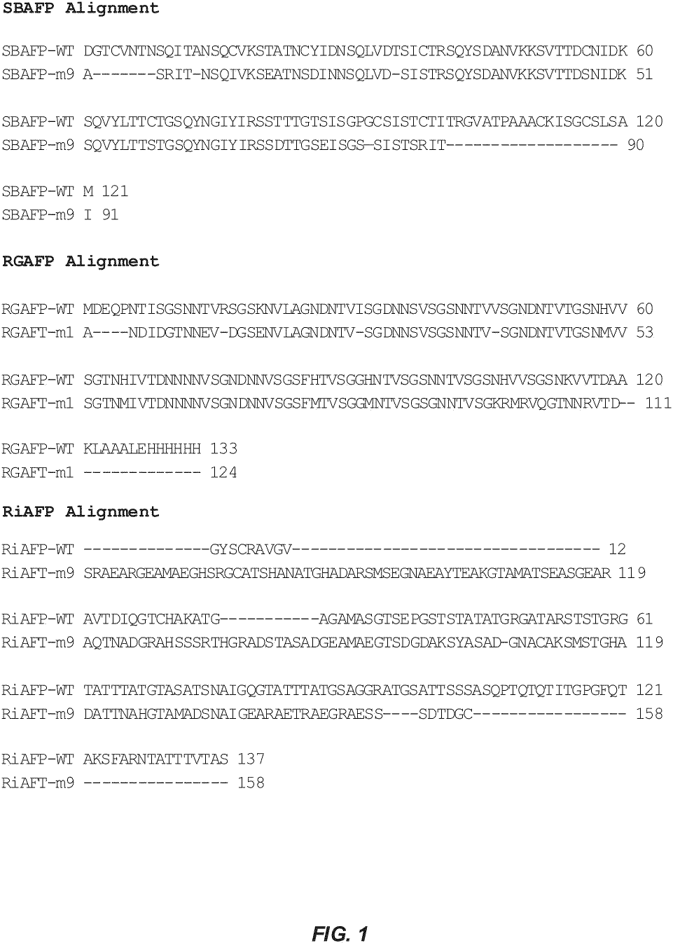

We propose development of direct virion capture assays based upon patented mBSP technology[5, see also 6-11] using genetically engineered, highly functionalizable beta solenoid proteins (FIGS. 1,2,3). Amino acid sequences allowing attachment to paper or nitrocellulose surfaces are well tolerated at the polypeptide termini, and the solenoid edges readily accept insertions that functionally mimic binding loops in antibodies.

Our approach to rapidly developing inexpensive SARS-CoV-2 POC direct viral capture tests will be two-fold. First, we provide a 54 residue helix-turn-helix (HTH) motif (SEQ ID NO:4 or a substantially similar sequence) from the cell-surface bound angiotensin converting enzyme 2 (ACE2) to which the SARS-CoV-2 viral spike protein binds. Second, phage display or other established biopanning technologies including ribosome display or yeast display can be used to find novel peptides that bind tightly and specifically to the SARS-CoV-2 spike protein.

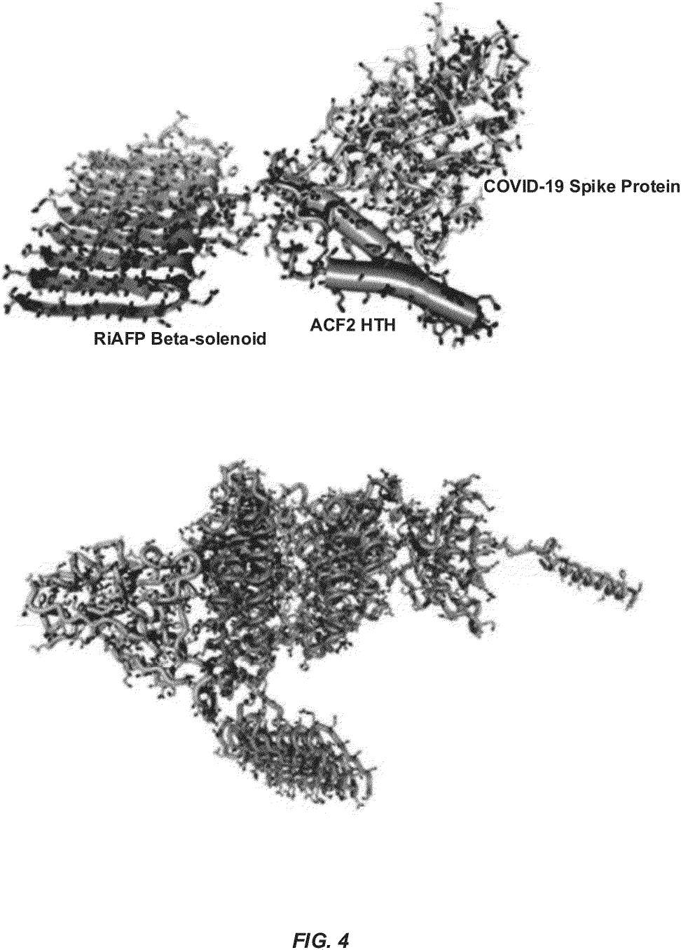

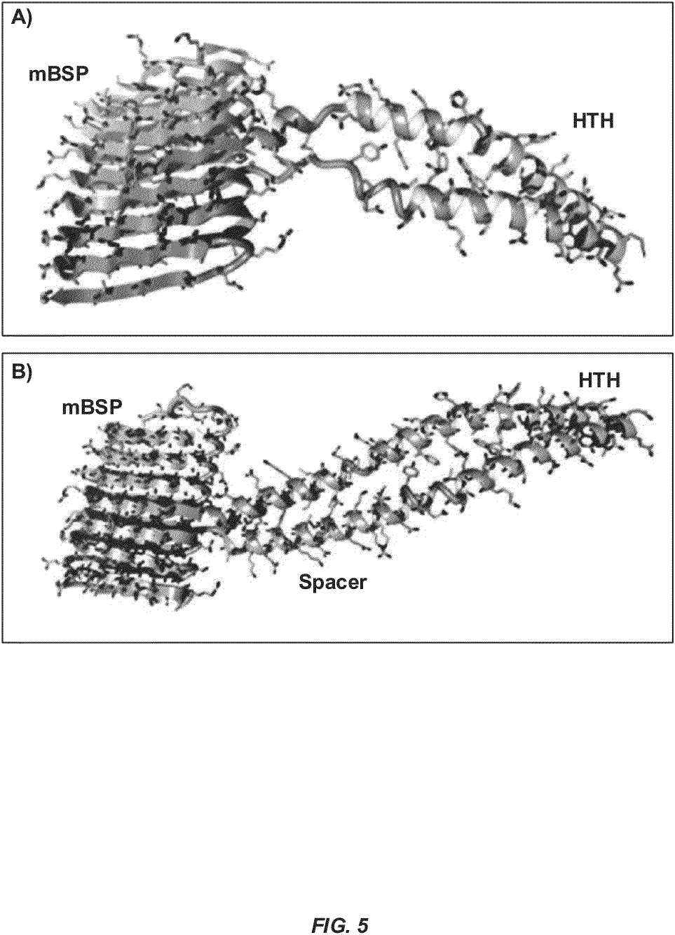

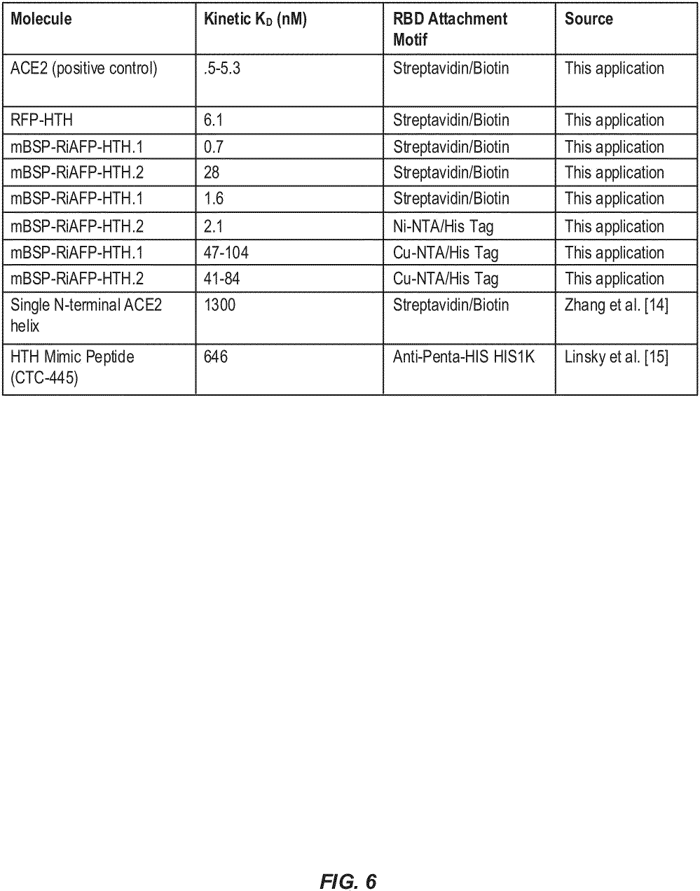

This represents a significant innovation. The cell-surface ACE2 protein [12] is the cellular entry point for SARS-CoV-2. ACE2 binds tightly to the SARS-CoV-2 spike protein, with dissociation constants in the low nanomolar range[13]. However, the single N-terminal helix of the ACE2 by itself binds weakly to the spike protein, with dissociation constants of 1.3 micromolar. [14], and a computationally designed substitute which includes a portion of the first helix only modestly improves on this with a dissociation constant of 646 nanomolar [15] We have made stable constructs of a VEGF-binding HTH fusion with RiAFP, discussed extensively in Example 2 below. Here we employ the ACE2 N-terminal HTH as a SARS-CoV-2 capture sequence (see below). This is illustrated in FIG. 4 and FIG. 5. The similarity of the HTH VEGF capture peptide with the ACE2 HTH sequence indicates the SARS-CoV-2 binding construct will function. That this use of HTH as a decoy binder is a non-obvious basis for success is seen in comparison of binding data for single RBDs to RiAFP-HTH.1 and RiAFP-.2 to single ACE2 proteins and to the single helix constructs above in FIG. 6. With the same binding motif (streptavidin-biotin) of RBD to a biolayer interferometry tip, we obtained dissociation constants in the range of 0.7-1.6 nM for the RiAFP-HTH.1 construct, and 2.1-28 nM for the RiAFP-HTH.2 construct. These are comparable to the 0.5-5.1 nM value we find for the ACE2 protein itself which are consistent with the literature (e.g., Ref. [13]).

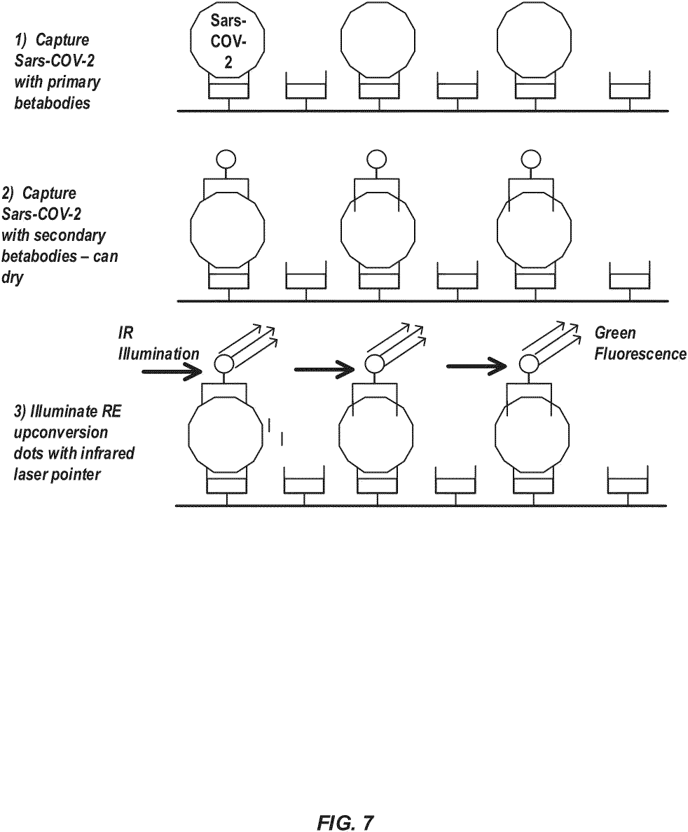

With a mBSP SARS-CoV-2 binding fusion construct in hand, a dense mBSP polymeric array is formed (FIG. 7, schematic) on suitable paper or polymer surfaces that can then capture the virus from patient fluids. Spectroscopic readout for virion capture will come from either (i) monomeric (end-capped) versions of the same mBSP SARS-CoV-2 binding fusion attached to luminescent nanoparticles, luminescent dyes or protein, or chromogenic reactions such as those catalyzed by horseradish peroxidase (as commonly employed in ELISA assays), or (ii) an environmentally sensitive dye introduced in a location that engenders fluorescence upon virion binding with no need for secondary mBSPs. Luminescence for (i) can be confirmed by illumination with LEDs or diode lasers of the appropriate output wavelength, and in (ii) by LED fluorescence excitation in the ~400 nm region and emission in the green (with, for example, 4-dimethylaminophthalimide-type fluorophores). Inexpensive hand-held fluorometers that can be modified for this application are commercially available or could rapidly be designed in collaboration with electrical engineers.

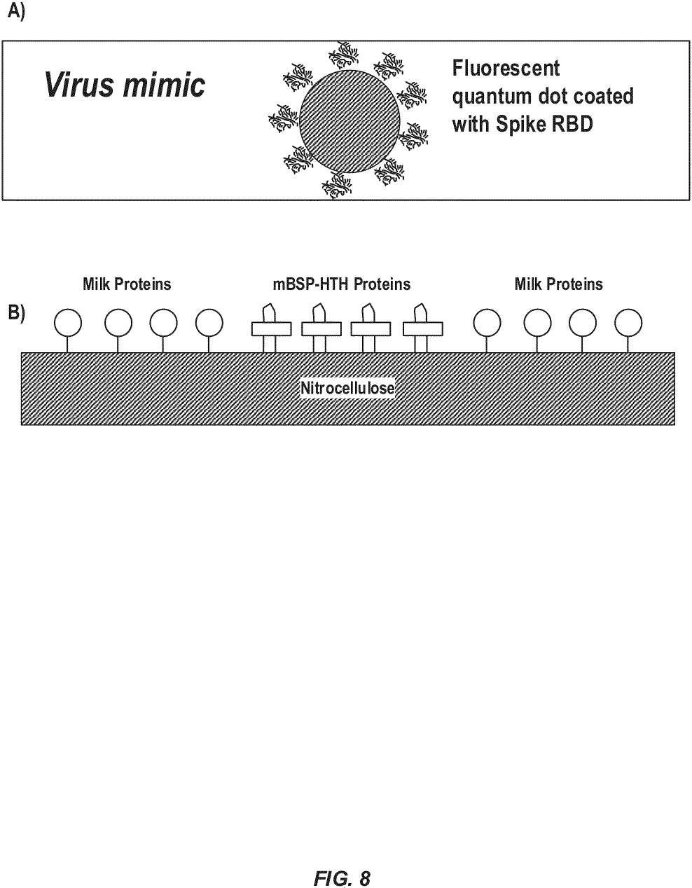

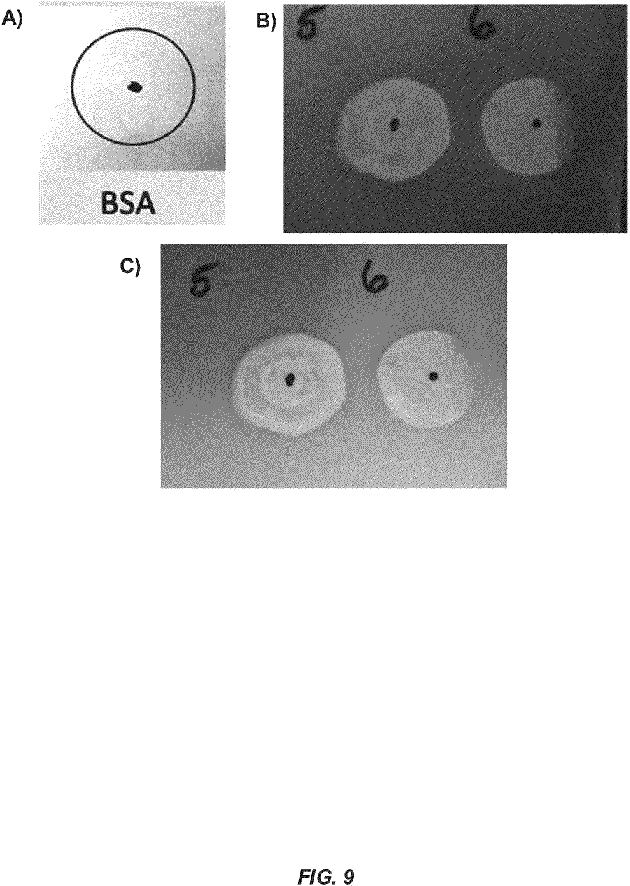

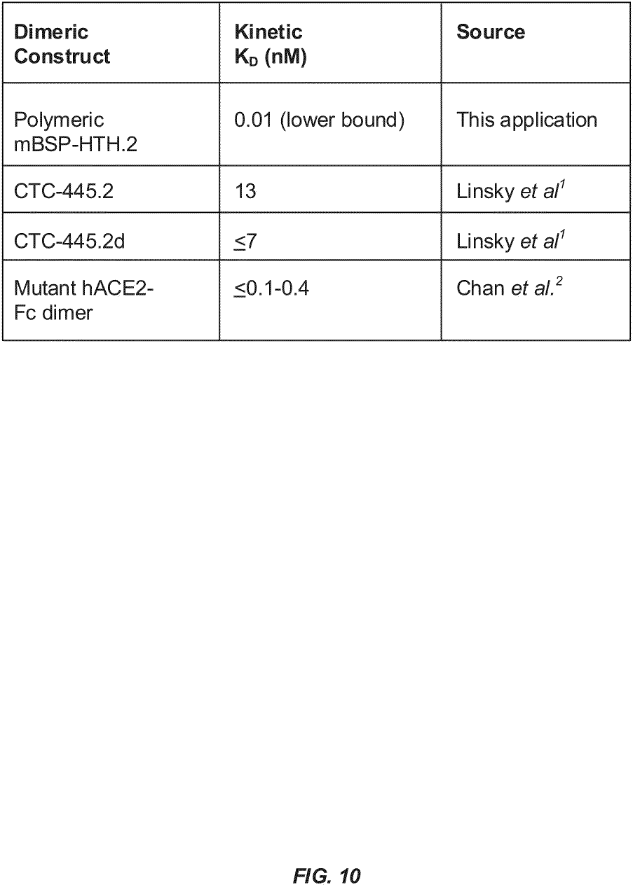

There are several built-in advantages to this mBSP based virion capture assay. First, it would likely be less expensive to employ than immunoassays and can potentially be quickly mass produced. Second, it requires no special technology and so can be implemented in field environments like drive-through test facilities. Third, it would likely detect the presence of either live or dead virus. Fourth, it is expected to be highly stable in extreme environments of heat or humidity. Our previous studies on mBSP polymers show stability in conditions of extreme alkalinity or acidity, high protein denaturant concentration, and extreme temperature (some survive autoclaving)[7]. They are however partially protease-sensitive, so do not present the same kind of potential health problems as say mammalian prions[16]. Fifth, it can be used for environmental sampling of viruses on surfaces. Sixth, a non-obvious consequence that will benefit the sensitivity and specificity of any assay based upon the polymeric capture proteins and a divalent conjugate labeling protein is at least divalent avidity compared to the monovalent binding of antibodies. FIG. 8 shows a schematic for a fluorescent assay to test for divalent binding of the capture polymers, and FIG. 9 shows results. In this experiment, proteins are spotted on a nitrocellulose surface and after the interstitial regions between spots are filled with milk proteins. Nanoparticles that fluoresce in the red are decorated by attaching single SARS-CoV-2 RBD proteins so that the mean spacing of ~7 nm matches the periodicity of the HTH constructs on the bound polymers. This implies at best dimeric binding of the RBDs. The nanoparticles are deposited on the surface, the surface is washed to clear stray particles and measurements are taken at two 24 hour intervals after washing. No intensity is seen on the Bovine Serum Albumin (BSA) control, and on two separate RiAFP-HTH.2 coated patches, fluorescent intensity is observed with a time decay rates of 6-9× 10⁻⁷ sec⁻¹. Using observed association rate constants of 10⁵ /sec-M gives dissociation constants of 6-9 pM, which we interpret as a lower bound of 10 pM. In contrast, the dimeric binding of the computational designed modified N-terminal fragments[15] and of dimeric ACE2 on an Fc dimer[17] give, respectively, binding affinities in the nM and tenths of nM. These values are summarized in FIG. 10.

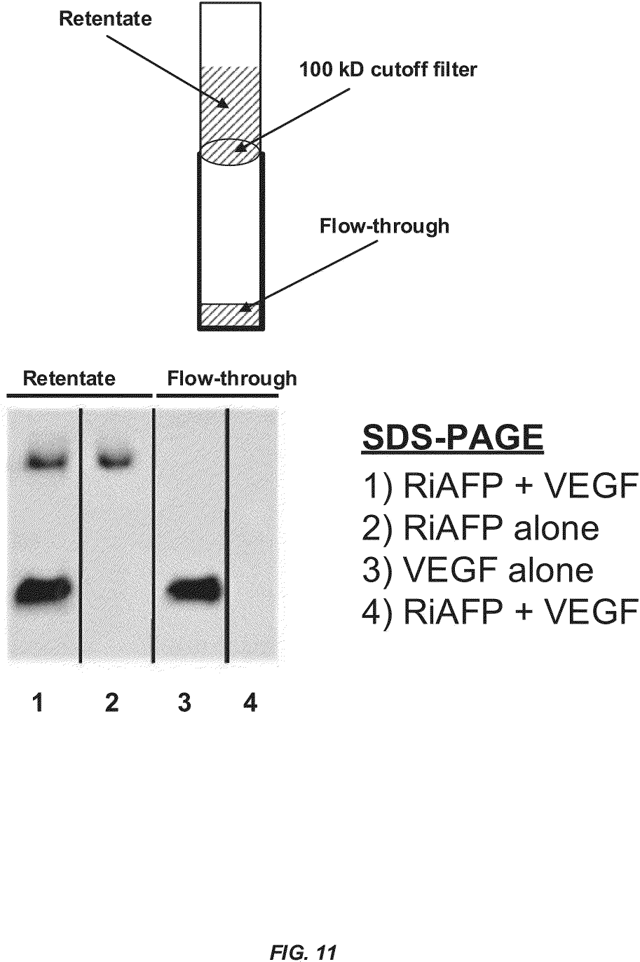

We have proven the ability of our mBSP-HTH constructs to bind VEGF by fusing a known VEGF binding HTH motif with mBSPs. We found tight binding in a simple assay based on ultrafiltration (FIG. 11). The calculated upper limit on the dissociation constant for VEGF binding is approximately 10 nM.

Development of secondary mBSPs bound to silica coated rare earth upconversion quantum nanoparticles (REUQN) for binding to RBD domains of SARS-CoV-2 spike proteins. REUQN make use of sequential two photon transitions from the infrared part of the spectrum followed by energy transfer to induce single photon luminescence in the visible spectrum; for example, one can use nanorings made from NaYF₄ doped with Er,Yb[18]. REUQN (i) can be excited by inexpensive infrared lasers[19], potentially enabling cell phone based imaging of binding, (ii) are stabilized with a silica coat to provide a shelf life of 4 years [18], (iii) can be functionalized for binding to amine or carboxyl functional groups, and (iv) are inexpensive to synthesize or purchase, and to coat. A previous antibody based FLISA assay for hepatitis B,C viruses made use of rare earth dots [20].

### Example 2

Construction and testing of betabodyBSP polymer comprising VEGF-binding protein: To prove that a BSP (beta solenoid based synthetic antibody [6] can be engineered to capture proteins with high affinity, we engineered a mBSP polymer derived from the modified Ragium Inquisitor antifreeze protein (RiAFP) [4] with a helix-tum-helix (HTH) motif known to bind to the Vascular Endothelial Growth Factor (VEGF) protein with high affinity [Fedorova, A., et al., The Development of Peptide-Based Tools for the Analysis of Angiogenesis. Chemistry & Biology, 2011. 18(7): p. 839-845]. We used a flow-through assay to verify that the mBSP polymer captured the VEGF with high affinity (K_{d} < 10 nM).

Experimental Design 1: Capture mBSPs We took a previously identified miniZ-peptide with the helix-turn-helix motif (HTH) that has approximately K_{d} = 40 nM affinity to VEGF. We designed and synthesized a construct with the miniZ-HTH inserted at the edge (FIG. 1A) of the mBSP, and we polymerized the resulting RiAFP-miniZ-HTH proteins (FIG. 1B).

Protein expression occurred from synthetic genes in the pET28a vector in E. coli followed by purification using standard methods. Polymerization was effected by incubating the proteins at 37° C. for 48 hours.

Experimental Design 2. We tested the binding of VEGF to the RiAFP-miniZ-HTH construct with a flow-through assay. The schematic is shown in FIG. 11. A centrifugal protein concentrator (Amicon) with a 100 kDa MW cutoff filter is centrifuged, with the samples initially placed in the upper (retentate) chamber. The filter blocks the RiAFP-miniZ-HTH polymers from flowing through because of the large size of the polymer, while allowing free VEGF to pass through. The contents of the retentate and flow-through are assessed using SDS-PAGE gel electrophoresis.

The samples analyzed were as follows:

I) Control 1: VEGF. This is shown in lane 3. After centrifugation, no VEGF is found in the retentate chamber, only in the flow-through chamber, confirming that it passes through the filter.

II) Control 2: RiAFP-miniZ-HTH. This is shown in lane 2. There is no protein found in the flow-through chamber, only in the retentate. This confirms that the filter blocks the RiAFP-miniZ-HTH construct.

III) Test sample. Both RiAFP-miniZ-HTH polymer and VEGF are added to the upper chamber prior to centrifugation. Both the RiAFP-miniZ-HTH polymer and VEGF appear only in the retentate, confirming VEGF binding by the polymer.

Analysis. The binding assay with controls demonstrates qualitatively high-affinity binding of VEGF to the RiAFP-miniZ-HTH construct. The limit of protein detection on SDS-PAGE gels stained with Coomassie R250 is generally accepted as ~30 ng. The SDS-PAGE gels were loaded with 20 µL of sample per lane. Thus, the test sample flow-through solution has less than 0.0015 mg/mL (~30 nM) VEGF. The concentrations of the RiAFP-miniZ-HTH and VEGF were each ~1 mg/mL (~40 µM RiAFP-miniZ-HTH; ~25 µM VEGF) in the initial mixture. Using the general equation for ligand binding combined with the values above allows us to estimate a Kd value of <10 nM for VEGF binding to RiAFP-miniZ-HTH.

The above examples are provided to illustrate the invention but not to limit its scope. Other variants of the invention will be readily apparent to one of ordinary skill in the art and are encompassed by the appended claims. All publications, databases, internet sources, patents, patent applications, and accession numbers cited herein are hereby incorporated by reference in their entireties for all purposes.

### REFERENCES

1. Aminat, F., et al., A serologic 1 al assay to detect SARS-CoV-2 seroconversion in humans. Nature Medicine 26, 1033-1036, 2020.

2. Woelfel, R., et al., Virological assessment of hospitalized patients with COVID-2019. Nature, 581, 465-469, 2020.

3. Venter, M., Richter, K., Towards effective diagnostic assays for COVID-19: a review, J. Clin. Pathol. 73, 370-377, 2020.

4. Hakim, A., et al., Crystal Structure of an Insect Antifreeze Protein and Its Implications for Ice Binding. Journal of Biological Chemistry, 2013. 288(17): p. 12295-12304.

5. Cox, D., et al., Self-assembled beta solenoid protein scaffolds U.S. Pat. US10287332B2, U.P. Office, Editor. 2019: USA.

6. M.D.R. Peralta, A.K., A. Ngo, C. Sierra, K. Fong, N.R. Hayre, N. Mirzaee, K. Ravikumar, A.J. Kluber, X. Chen, G.Y. Liu, M.D. Toney, R.R.P. Singh, and D.L. Cox Engineering Amyloid Fibrils from β-Solenoid Proteins for Biomaterials Applications, ACS Nano 9, 449-463, 2020.

7. Peng, Z., M.D.R. Peralta, and M.D. Toney, Extraordinarily Stable Amyloid Fibrils Engineered from Structurally Defined beta-Solenoid Proteins. Biochemistry, 2017. 56(45): p. 6041-6050.

8. Peng, Z., et al., High Tensile Strength of Engineered beta-Solenoid Fibrils via Sonication and Pulling. Biophysical Journal, 2017. 113(9): p. 1945-1955.

9. Baard, R.A., et al., In silico stress-strain measurements on self-assembled protein lattices. Soft Matter, 2018. 14(40): p. 8095-8104.

10. Heinz, L.P., K.M. Ravikumar, and D.L. Cox, In Silico Measurements of Twist and Bend Moduli for beta-Solenoid Protein Self-Assembly Units. Nano Letters, 2015. 15(5): p. 3035-3040.

11. Peng, Z., et al., Bottom-up synthesis of protein-based nanomaterials from engineered beta-solenoid proteins. PloS one, 2020. 15(2): p. e0229319-e0229319.

12. Tai, W., et al., Characterization of the receptor-binding domain (RBD) of 2019 novel coronavirus: implication for development of RBD protein as a viral attachment inhibitor and vaccine. Cellular & molecular immunology 17, 613-620, 2020.

13. Walls, A.C., et al., Structure, Function, and Antigenicity of the SARS-CoV-2 Spike Glycoprotein. Cell 181, 281-292 2020.

14. G. Zhang, S. Pomplun, A.R. Loftis, X. Tan, A. Loas, and B.L. Pentelute, “Investigation of ACE2 N-terminal fragments binding to SARS-CoV-2 Spike RBD”, bioRxiv.org, https://doi.org/10.1101/2020.03.19.999318, (2020).

15. T.W. Linsky et al., “De novo design of potent and resilient hACE2 decoys to neutralize SARS-CoV-2,” Science 370, 1208-1214 (2020).

16. Forloni, G., et al., NEUROTOXICITY OF A PRION PROTEIN-FRAGMENT. Nature, 1993. 362(6420): p. 543-546.

17. K.K. Chan, T.J.C.Tan, K.K. Narayanan, E. Procko, “An engineered decoy receptor for SARS-CoV-2 broadly binds S sequence variants,” Science Advances 7, eabf1738 (2021).

18. Mullen, T.J., et al., Fabrication and Characterization of Rare-Earth-Doped Nanostructures on Surfaces. Acs Nano, 2011. 5(8): p. 6539-6545.

19. Inc, L. Focusable <5mw 980 nm IR Infrared Laser Pointer Point Pen. 2017; Available from: https://www.laserlands.net/focusable-5mw-980nm-ir-infrared-laser-pointer-point-pen.html?gclid=CjwKCAjw87PNBRBAEiwAOXAlr-ZUr-U3RCii0x39VtXllmGx6QTZHvsTpf2-naadlf3RwvOpJm8TdhoCf2MQAvD_BwE.

20. Talha, S.M., et al., Europium nanoparticle-based simple to perform dry-reagent immunoassay for the detection of hepatitis B surface antigen. Journal of Virological Methods, 2016. 229: p. 66-69.

21. Zhuang, Y.-D., et al., Environment-Sensitive Fluorescent Turn-On Probes Targeting Hydrophobic Ligand-Binding Domains for Selective Protein Detection. Angewandte Chemie-International Edition, 2013. 52(31): p. 8124-8128.

1. An amyloid fibril comprising a plurality of modified β solenoid protein (mBSP) monomers, wherein the monomers are linked to a pathogen-binding protein.

2. The amyloid fibril of claim 1, wherein the mBSP monomers are derived from an antifreeze protein.

3. The amyloid fibril of claim 2, wherein the antifreeze protein is a spruce budworm antifreeze protein.

4. The amyloid fibril of claim 3, wherein the mBSP has the sequence shown in SEQ ID NO: 1 or a sequence at least 90% identical to SEQ ID NO:1.

5. The amyloid fibril of claim 1, wherein the antifreeze protein is a rye grass antifreeze protein.

6. The amyloid fibril of claim 5, wherein the mBSP has the sequence shown in SEQ ID NO: 2 or a sequence at least 90% identical to SEQ ID NO:2.

7. The amyloid fibril of claim 1, wherein the antifreeze protein is a rhagium inquisitor antifreeze protein.

8. The amyloid fibril of claim 7, wherein the mBSP has the sequence shown in SEQ ID NO: 3 or a sequence at least 90% identical to SEQ ID NO: 3.

9. The amyloid fibril of any of claim 1, wherein the mBSP is modified to remove an end cap that prevents amyloid aggregation.

10. The amyloid fibril of claim 1 that is modified to include at least one amino acid residue that promotes attachment of the fibril to a solid support, a nanoparticle, a biological molecule, or a second amyloid fibril.

11. The amyloid fibril of claim 1, attached to a solid support, a nanoparticle, a biological molecule, or a second amyloid fibril.

12. The amyloid fibril of claim 1, wherein the pathogen is a virus.

13. The amyloid fibril of claim 11, wherein the virus is SARS-COV-2.

14. The amyloid fibril of claim 1, wherein the amyloid fibril is linked to a solid support.

15. (canceled)

16. (canceled)

17. The amyloid fibril of claim 1, wherein the pathogen-binding protein comprises the N-terminal ACE2 helix-turn-helix (HTH) domain (e.g., SEQ ID NO: 4, or a sequence at least 90% identical to SEQ ID NO:4).

18. The amyloid fibril of claim 1, wherein the pathogen binding protein is linked to a detectable label.

19. (canceled)

20. A method of detecting the presence or absence of a pathogen in a biological sample, the method comprising contacting a biological sample with the amyloid fibril of claim 1 under conditions that allow the pathogen to bind to the pathogen binding protein if the pathogen is present; and detecting the presence or absence of binding of the pathogen to the amyloid fibril, wherein the detecting comprises washing unbound components of the sample from the amyloid fibril and contacting the amyloid fibril, and bound pathogen if present, with a secondary binding agent that specifically binds to the pathogen, if bound to the amyloid fibril, and wherein the secondary binding agent is a β solenoid protein linked to a pathogen binding protein.

21. (canceled)

22. (canceled)

23. (canceled)

24. The method of claim 20, wherein the secondary binding agent is linked to a detectable label.

25. The method of claim 20, wherein the β solenoid protein comprises a sequence that prevents polymerization.

26. Clothing or protective equipment coated with the amyloid fibril of claim 1.


SEQUENCE LISTING (21 sequences)

SEQ ID NO: 1 - protein, 91 aa, Artificial Sequence
  Description of Artificial Sequence: Synthetic polypeptide
ASRITNSQIVKSEATNSDINNSQLVDSISTRSQYSDANVKKSVTTDSNIDKSQVYLTTSTGSQYNGIYIRSSDTTGSEISGSSISTSRITI

SEQ ID NO: 2 - protein, 111 aa, Artificial Sequence
  Description of Artificial Sequence: Synthetic polypeptide
ANDIDGTNNEVDGSENVLAGNDNTVSGDNNSVSGSNNTVSGNDNTVTGSNMVVSGTNMIVTDNNNNVSGNDNNVSGSFMTVSGGMNTVSGSNNTVSGKRMRVQGTNNRVTD

SEQ ID NO: 3 - protein, 158 aa, Artificial Sequence
  Description of Artificial Sequence: Synthetic polypeptide
SRAEARGEAMAEGHSRGCATSHANATGHADARSMSEGNAEAYTEAKGTAMATSEASGEARAQTNADGRAHSSSRTHGRADSTASAKGEAMAEGTSDGDAKSYASADGNACAKSMSTGHADATTNAHGTAMADSNAIGEARAETRAEGRAESSSDTDGC

SEQ ID NO: 4 - protein, 63 aa, Unknown
  Description of Unknown: Helix-turn-helix motif sequence
IEEQAKTFLDKFNHEAEDLFTQSSLASTNTNTNITEENVQNMNNAGDKTSAFLKEQSTLAQMT

SEQ ID NO: 5 - protein, 233 aa, Artificial Sequence
  Description of Artificial Sequence: Synthetic polypeptide
ASRAEARGEAMAEGHSRGSATSHANATGHADARSMSEGNAEAYTEAKGDAMATSEASGEARAQTNADGSAHSSSRTHGRADSTASAKTNYNRECGEEQAKTFLDKFNHEAEDLFYQSSLASWNYNTNITEENVQNRNNAGDKRSAFLKEQSTLAQMYGCGSGSAMAEGTSDGDAKSYASADGNASAKSMSTGHADATTNAHGTAMADSNAIGEARAETRAEGRAESSSDTDGS

SEQ ID NO: 6 - protein, 266 aa, Artificial Sequence
  Description of Artificial Sequence: Synthetic polypeptide
ASRAEARGEAMAEGHSRGSATSHANATGHADARSMSEGNAEAYTEAKGDAMATSEASGEARAQTNADGSAHSSSRTHGRADSTASAKGGKALNDKEAKNKAILNLEEIEEQAKTFLDKFNHEAEDLFYQSSLASWNYNTNITEENVQNMNNAGDKWSAFLKEQSTLAQMYQNEIKRKSEKQEDLKKEMLELEKLGGAMAEGTSDGDAKSYASADGNASAKSMSTGHADATTNAHGTAMADSNAIGEARAETRAEGRAESSSDTDGS

SEQ ID NO: 7 - protein, 121 aa, Unknown
  Description of Unknown: Choristoneura fumiferana
DGTCVNTNSQITANSQCVKSTATNCYIDNSQLVDTSICTRSQYSDANVKKSVTTDCNIDKSQVYLTTCTGSQYNGIYIRSSTTTGTSISGPGCSISTCTITRGVATPAAACKISGCSLSAM

SEQ ID NO: 8 - protein, 133 aa, Lolium perenne
MDEQPNTISGSNNTVRSGSKNVLAGNDNTVISGDNNSVSGSNNTVVSGNDNTVTGSNHVVSGTNHIVTDNNNNVSGNDNNVSGSFHTVSGGHNTVSGSNNTVSGSNHVVSGSNKVVTDAAKLAAALEHHHHHH

SEQ ID NO: 9 - protein, 137 aa, Rhagium inquisitor
GYSCRAVGVDGRAVTDIQGTCHAKATGAGAMASGTSEPGSTSTATATGRGATARSTSTGRGTATTTATGTASATSNAIGQGTATTTATGSAGGRATGSATTSSSASQPTQTQTITGPGFQTAKSFARNTATTTVTAS

SEQ ID NO: 10 - protein, 20 aa, Unknown
  Description of Unknown: Choristoneura fumiferana
RGVATPAAACKISGCSLSAM

SEQ ID NO: 11 - protein, 15 aa, Lolium perenne
AAKLAAALEHHHHHH

SEQ ID NO: 12 - protein, 19 aa, Rhagium inquisitor
GYSCRAVGVDGRAVTDIQG

SEQ ID NO: 13 - protein, 20 aa, Rhagium inquisitor
QPTQTQTITGPGFQTAKSFA

SEQ ID NO: 14 - protein, 184 aa, Unknown
  Description of Unknown: Cyanothece sp.
MHHHHHHSSGLVPRGSGMKETAAKFERQHMDSPDLGTDDDDKAMAMVTGSSASYEDVKLIGEDFSGKSLTYAQFTNADLTDSNFSEADLRGAVFNGSALIGADLHGADLTNGLAYLTSFKGADLTNAVLTEAIMMRTKFDDAKITGADFSLAVLDVYEVDKLCDRADGVNPKTGVSTRESLRCQ

SEQ ID NO: 15 - protein, 257 aa, Nostoc sp.
MGSSHHHHHHSSGLVPRGSHMNVGEILRHYAAGKRNFQHINLQEIELTNASLTGADLSYADLRQTRLGKSNFSHTCLREADLSEAILWGIDLSEADLYRAILREADLTGAKLVKTRLEEANLIKASLCGANLNSANLSRCLLFQADLRPSSNQRTDLGYVLLTGADLSYADLRAASLHHANLDGAKLCRANFGRTIQWGNLAADLSGASLQGADLSYANLESAILRKANLQGADLTGAILKDAELKGAIMPDGSIHD

SEQ ID NO: 16 - protein, 124 aa, Artificial Sequence
  Description of Artificial Sequence: Synthetic polypeptide
MKQFTKCELSQLLKDIDGYGGIALPELICTMFHTSGYDTQAIVENNESTEYGLFQISNKLWCKSSQVPQSRNICDISCDKFLDDDITDDIMCAKKILDIKGIDYWLAHKALCTEKLEQWLCEKL

SEQ ID NO: 17 - protein, 334 aa, Unknown
  Description of Unknown: Salmonella enterica
  MOD_RES (1)..(1): Any amino acid
  MOD_RES (67)..(67): Any amino acid
XTKYRLSEGPRAFTYQVDGEKKSVLLRQVIAVTDFNDVKAGTSGGWVDADNVLSQQGDCWIYDENAXAFAGTEITGNARITQPCTLYNNVRIGDNVWIDRADISDGARISDNVTIQSSSVREECAIYGDARVLNQSEILAIQGLTHEHAQILQIYDRATVNHSRIVHQVQLYGNATITHAFIEHRAEVFDFALIEGDKDNNVWICDCAKVYGHARVIAGTEEDAIPTLRYSSQVAEHALIEGNCVLKHHVLVGGHAEVRGGPILLDDRVLIEGHACIQGEILIERQVEISGRAAVIAFDDNTIHLRGPKVINGEDRITRTPLVGSLLEHHHHHH

SEQ ID NO: 18 - protein, 7 aa, Unknown
  Description of Unknown: Hepatitis B binding protein sequence
ETGAKPH

SEQ ID NO: 19 - protein, 7 aa, Unknown
  Description of Unknown: Hepatitis C binding protein sequence
TSQNIRS

SEQ ID NO: 20 - protein, 7 aa, Unknown
  Description of Unknown: Hepatitis C binding protein sequence
WPWHNHR

SEQ ID NO: 21 - protein, 12 aa, Unknown
  Description of Unknown: Hepatitis C binding protein sequence
MARHRNWPLVMV